(19) United States
(12) Patent Application Publication
(10) Pub. No.: US 2011/0171709 A1
(43) Pub. Date: Jul. 14, 2011

(54) Product Recovery From Fermentation of Lignocellulosic Biomass

(75) Inventor: John Bardsley, Newport, NH (US)

(73) Assignee: Mascoma Corporation

(21) Appl. No.: 12/740,839

(22) PCT Filed: Oct. 29, 2008
(86) PCT No.: PCT/US08/12239

§ 371 Date: Dec. 6, 2010

Related U.S. Application Data
(60) Provisional application No. 60/996,094, filed on Nov. 1, 2007.

(51) Int. Cl. C12P 7/10

(52) U.S. Cl. 435/165

(57) ABSTRACT

The present invention is directed to a process of producing ethanol from lignocellulosic biomass, which comprises pre-treating a lignocellulosic feedstock to produce a reactive carbohydrate mixture; adding activated carbon in free form; converting said reactive carbohydrate mixture to form a beer; separating solids from said carbohydrate mixture or said beer or both, wherein said activated carbon is separated along with the solids in said mixture, said beer or both; and drying said solids. The invention is also directed to the production of a dried solid fuel to be combusted during said process.

### BACKGROUND OF THE INVENTION

1. Field of the Invention

The present invention is directed to processes for producing ethanol from lignocellulosic biomass. In one aspect the process rely upon adding activated carbon in free form; the activated carbon is separated along with byproduct solids. The invention is also directed to the production of a dried solid fuel to be combusted during said process.

2. Background Art

Plant biomass and derivatives thereof are a natural resource for the biological conversion of energy to forms useful to humanity. Among forms of plant biomass, lignocellulosic biomass is particularly well-suited for energy applications because of its large-scale availability, low cost, and environmentally benign production. In particular, many energy production and utilization cycles based on lignocellulosic biomass have near-zero greenhouse gas emissions on a life-cycle basis.

Ethanol is the primary biologically-derived transportation fuel worldwide, with production mainly from corn in the U.S. and from sugarcane in Brazil. Domestic ethanol production currently decreases oil imports, reduces greenhouse gas emissions, and increases farm income, reducing federal crop support expenditures. The economics of corn ethanol production have been attractive over the last several years due to a combination of factors including low corn prices, high crude oil prices, technological improvements from over two decades of commercial production, government incentives, stable co-product prices, and demand stimulated by the renewable fuel standard passed as part of the energy policy act of 2005. With potential for two year investor payback periods on corn ethanol plants, the industry build-out has been bullish and production capacity has risen sharply from 3.6 billion gallons in 2004 to 5.1 billion gallons in the fall of 2006, with 3.6 billion gallons of additional capacity under construction. In 2006, ethanol production consumed 20% of the U.S. corn crop, and accounted for about 2% of U.S. fuel consumption for light-duty vehicles.

The rapid growth of the industry, however, has increased demand for corn, and as a result corn prices have risen from an average of $2.30 per bushel over the last 5 years, and $1.95 per bushel in 2006, to over $3.50 per bushel in the spring of 2007. While high corn prices are advantageous for corn growers, they reduce the profitability of ethanol production as well as other agricultural activities that consume corn, such as pork, animal feed, and poultry production. Moreover, environmental advocacy organizations, such as the NRDC and World Wildlife Fund, are concerned about the water quality and soil fertility implications of increased corn planting.

Independent of the status and future prospects of the corn ethanol industry, ethanol production from cellulosic biomass, such as wood, grass, and agricultural residues, has attracted a great deal of attention of late. Although cellulosic ethanol is not yet produced commercially, projected features include a decisively positive fossil fuel displacement ratio, near-zero net greenhouse gas emissions, potential for substantial soil fertility and carbon sequestration benefits, and feedstocks with broad geographical diversity, expected to be widely available at a cost per unit energy (e.g. $17/GJ) equal to that provided by oil were it available at about $17/barrel.

Several studies foresee the possibility of cellulosic ethanol playing a large role in meeting national mobility demands, particularly when combined with improved vehicle efficiency. As noted above, cellulosic ethanol is not widely produced commercially in the at the current time, but efforts to commercialize both biological and thermo-chemical processes are underway.

Thermo-chemical processes use heat, pressure, and steam to convert feedstock into synthesis gas (“syngas”). Syngas is passed over a catalyst and transformed into alcohols such as ethanol. Biological processes to convert cellulosic biomass into ethanol involve pretreatment, production of reactive carbohydrate, and biological conversion, in which the carbohydrate is converted into ethanol. The beer output from biological conversion contains ethanol and non-fermented solids, which are both recovered for storage and sale in downstream processing.

The primary obstacle impeding the more widespread production of energy from biomass feedstocks is the general absence of low-cost technology for overcoming the recalcitrance of these materials. As outlined above, cellulosic ethanol can be produced from a wide variety of cellulosic biomass feedstocks including agricultural plant wastes (corn stover, cereal straws, sugarcane bagasse), plant wastes from industrial processes (sawdust, paper pulp), consumer waste and energy crops grown specifically for fuel production, such as switchgrass. Cellulosic biomass is composed of, cellulose, hemicellulose and lignin, with smaller amounts of proteins, lipids (fats, waxes and oils) and ash. Roughly, two-thirds of the dry mass of cellulosic materials are present as cellulose and hemicellulose. Lignin makes up the bulk of the remaining dry mass.

The production of ethanol from biomass typically involves the breakdown or hydrolysis of lignocellulose-containing materials into disaccharides and, ultimately, monosaccharides. Processing cellulosic biomass aims to extract fermentable sugars from the feedstock. The sugars in cellulose and hemicellulose are locked in complex carbohydrates called polysaccharides (long chains of monosaccharides or simple sugars). Separating these complex polymeric structures into fermentable sugars is essential to the efficient and economic production of cellulosic ethanol.

A number of processing options are employed to produce fermentable sugars from cellulosic biomass. One approach utilizes acid hydrolysis to break down the complex carbohydrates into simple sugars. An alternative method, enzymatic hydrolysis, utilizes pretreatment processes to first reduce the size of the material to make it more accessible to hydrolysis. Once pretreated, enzymes are employed to convert the cellulosic biomass to fermentable sugars. The final step involves microbial fermentation yielding ethanol and carbon dioxide.

However, cellulosic ethanol production presents a number of challenges that must be met in order to economically and efficiently produce ethanol from biomass. During the course of biomass pre-treatment, degradation products are formed that act as fermentation inhibitors. Longer treatment times and lower yields result. As another example, challenges exist in the removal of solids from the production stream of cellulosic ethanol. In the production of alcohol from plant materials, the biomass is mixed with hot water to produce a wort, which is fermented until the final alcohol level is reached. The fermented contents are then typically discharged as a slurry (“beer”) to the beer well and from there to the still where the alcohol is removed by distillation. The remainder, after distillation, is non-fermented insoluble material known as “stillage,” and consists of a large amount of water together with the solids. However, the solids concentration in cellulosic beer is high and also contain soluble pentose and hexose sugars that first-generation organisms deployed in cellulosic ethanol processes are unable to metabolize.

It is therefore necessary to maximize the pre-treatment, hydrolysis and promote the fermentation of all available carbohydrates to maximize ethanol yield in lignocellulosic fermentation methods. As such, the addition of activated carbon to the reactive carbohydrate mixture, during the process of producing ethanol from lignocellulose biomass can remove chemical inhibitors of fermentation, thereby increasing the efficiency and yield of ethanol produced from lignocellulosic biomass.

### BRIEF SUMMARY OF THE INVENTION

The present invention is directed to a process of producing ethanol from lignocellulosic biomass, which comprises: pre-treating a lignocellulosic feedstock to produce a reactive carbohydrate mixture; adding activated carbon in free form; converting said reactive carbohydrate mixture to form a beer; separating solids from said carbohydrate mixture or said beer or both, wherein said activated carbon is separated along with the solids in said mixture, said beer or both; and drying said solids.

In certain embodiments of the process of the present invention, converting can be chemically converting or biologically converting said reactive carbohydrate mixture to form a beer. Certain embodiments of the process of the present invention further comprise: separating activated carbon and solids remaining after pre-treating or solids remaining after said biological conversion.

In some further embodiments of the process of the present invention, separating of said activated carbon and solids is selected from the group consisting of beer column tray separation, paddle dryer apparatus separation, twin screw conveyer separation, rotary valve separation, butterfly valve separation, distillation, centrifuging and combinations thereof.

In certain embodiments of the process of the present invention further comprises de-watering, drying directly or indirectly, and pressing said activated carbon and solids to form a dried solid cake. In other embodiments of the process of the present invention further comprises combusting said dried solid cake to provide heat during production of said ethanol from said lignocellulosic biomass.

In certain embodiments of the present invention lignocellulosic biomass is selected from the group consisting of grass, switch grass, cord grass, rye grass, reed canary grass, miscanthus, mixed prairie grasses, sugar-processing residues, sugarcane bagasse, agricultural wastes, rice straw, rice hulls, barley straw, corn cobs, cereal straw, wheat straw, canola straw, oat straw, oat hulls, beet pulp, palm residue, corn fiber, stover, soybean stover, corn stover, forestry wastes, recycled wood pulp fiber, paper sludge, sawdust, hardwood, softwood, and combinations thereof.

In certain embodiments of the process of the present invention, pre-treating is selected from the group consisting of catalytic treatment, acid treatment, alkaline treatment, organic solvent treatment, steam treatment, heat treatment, low-pH treatment, pressure treatment, milling treatment, steam explosion treatment, pulping treatment or white rot fungi treatment and combinations thereof, in further embodiments the pre-treatment is a combination of steam treatment and heat treatment.

In certain embodiments of the process, said converting comprises hydrolyzing cellulose and hemi-cellulose to form monomeric sugars, oligosaccharides, or a combination thereof, and fermenting said monomeric sugars, oligosaccharides, or a combination thereof to produce ethanol.

In some further embodiments of the present invention, hydrolyzing comprises enzymatically hydrolyzing cellulose and hemi-cellulose to form monomeric sugars, in certain embodiments, said hydrolyzing comprises chemically hydrolyzing cellulose and hemi-cellulose to form monomeric sugars.

In certain embodiments, said hydrolyzing and fermenting occur concurrently in the same reactor and in certain embodiments of the present invention hydrolyzing and fermenting are concurrent and occur in the presence of activated carbon in free form and in some further embodiments, said activated carbon is granulated or powdered.

In certain embodiments of the present invention said dried solid cake comprises about 1% to about 30% activated carbon and in certain embodiments the process of present invention further comprises burning said dried solid cake as a fuel, wherein said fuel contains about 1 BTU per kilogram to about 16,500 BTU per kilogram. In certain embodiments of the present invention, said dried solid cake comprises activated carbon, lignin, cellulosic sugars, ethanol, water and combinations thereof.

In further embodiments of the present invention, the addition of about 1% to about 6% activated carbon in free form increases the amount of ethanol produced by said process about 50% to about 200% in about 24 hours.

### BRIEF DESCRIPTION OF THE DRAWINGS

FIG. 1 depicts an example of a system for processing of lignocellulosic biomass to ethanol.

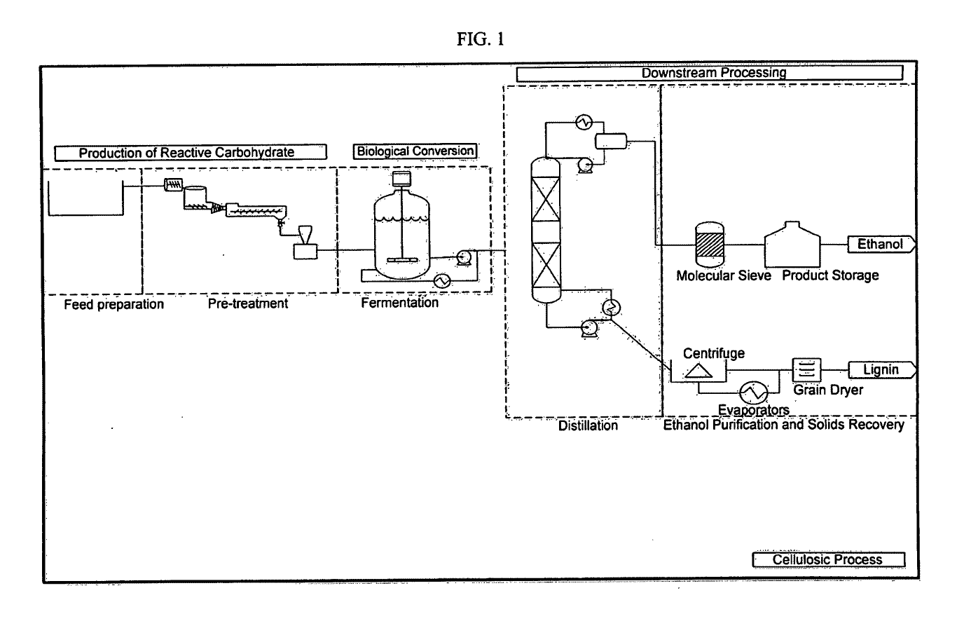

FIG. 2 depicts the relationship between the addition of 1% activated carbon and ethanol output from concurrent hydrolysis and fermentation of a pretreated sample of 30% MS029 lignocellulosic biomass over 24 hours.

### DETAILED DESCRIPTION OF THE INVENTION

The present invention is directed to a process of producing ethanol from lignocellulosic biomass, which comprises: pre-treating a lignocellulosic feedstock to produce a reactive carbohydrate mixture; adding activated carbon in free form; converting said reactive carbohydrate mixture to form a beer; separating solids from said carbohydrate mixture or said beer or both, wherein said activated carbon is separated along with the solids in said mixture, said beer or both; and drying said solids.

Biomass can be classified in three main categories: sugar, starch and cellulose containing plants. Cellulose-containing plants and waste products are the most abundant forms of biomass, such materials are referred to as lignocellulosic biomass because they contain cellulose (20% to 60%), hemicellulose (10% to 40%) and lignin (5% to 25%) whilst non-woody biomass generally contains less than about 15-20% lignin.

The terms “hemicellulose,” “hemicellulosic portions,” and “hemicellulosic fractions” mean the non-lignin, non-cellulose elements of lignocellulosic material, such as but not limited to hemicellulose (comprising xyloglucan, xylan, glucuronoxylan, arabinoxylan, mannan, glucomannan, and galactoglucomannan, inter alia), pectins (e.g., homogalacturonans, rhamnogalacturonan I and II, and xylogalacturonan), and proteoglycans (e.g., arabinogalactan-protein, extensin, and proline-rich proteins).

In certain embodiments lignocellulosic biomass can include, but is not limited to, woody biomass, such as recycled wood pulp fiber, sawdust, hardwood, softwood, and combinations thereof; grasses, such as switch grass, cord grass, rye grass, reed canary grass, miscanthus, mixed prairie grasses, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, beet pulp; palm residue, corn fiber, and stover, such as but not limited to soybean stover, corn stover; and forestry wastes, such as but not limited to recycled wood pulp fiber, sawdust, hardwood (e.g., poplar, oak, maple, birch), softwood, or any combination thereof.

Paper sludge is also a viable feedstock for ethanol production. Paper sludge is solid residue arising from pulping and paper-making, and is typically removed from process wastewater in a primary clarifier. The size range of the substrate material varies widely and depends upon the type of substrate material used as well as the requirements and needs of a given process. In certain embodiments of the invention, the lignocellulosic biomass may be prepared in such a way as to permit ease of handling in conveyors, hoppers and the like. In the case of wood, the chips obtained from commercial chippers are suitable; in the case of straw it is sometimes desirable to chop the stalks into uniform pieces about 1 to about 3 inches in length. Depending on the intended degree of pretreatment, the size of the substrate particles prior to pretreatment may range from less than a millimeter to inches in length.

Cellulose molecules are linear, unbranched and can have polymerization ranges from 500 to 20,000 and have a strong tendency to form inter- and intra-molecular hydrogen bonds. Bundles of cellulose molecules are thus aggregated together to form microfibrils in which highly ordered (crystalline) regions alternate with less ordered (amorphous) regions. Microfibrils make fibrils and finally cellulose fibers. As a consequence of its fibrous structure and strong hydrogen bonds, cellulose has a very high tensile strength and is insoluble in most solvents.

Lignocellulosic biomass must therefore undergo pre-treatment to enhance susceptibility to hydrolysis. The degradation of lignocellulosics is primarily governed by its structural features because cellulose possesses a highly ordered structure and the lignin surrounding cellulose forms a physical barrier.

Pretreatment is required to reduce the lignin content, reduce the order of the cellulose and increases surface area. Pretreatment methods can be physical, chemical, physicochemical and biological, depending on the mode of action. The various pretreatment methods that have been used to increase cellulose digestibility include ball-milling treatment, two-roll milling treatment, hammer milling treatment, colloid milling treatment, high pressure treatment, radiation treatment, pyrolysis, catalytic treatment, acid treatment, alkaline treatment, organic solvent treatment, steam treatment, heat treatment, low-pH treatment, steam explosion treatment, pulping treatment, white rot fungi treatment, steam explosion and ammonia fiber explosion and combinations thereof. A further discussion of pretreatments can be found in Holtzapple et al. (U.S. Pat. No. 5,865,898; hereby incorporated by reference). Exposure time, temperature, and pH are the additional metrics that govern the extent to which the cellulosic carbohydrate fractions are cleaved during pre-treatment and amenable to further enzymatic hydrolysis in subsequent biological conversion steps.

In certain embodiments physicochemical pretreatment is Ammonia Fiber Explosion (AFEX). AFEX requires soaking the lignocellulose in liquid ammonia at high pressure, followed by an explosive release of the pressure. Pretreatment conditions (about 30° C. to about 100° C.) are less severe than steam explosion. An increase in accessible surface area coupled with reduced cellulose crystallinity (caused by ammonia contacting) result in increased enzymatic digestibility. For example, the use of ammonia under pressure to increase protein availability and cellulosic digestibility of a cellulosic containing plant material (alfalfa) is described in Hultquist (U.S. Pat. No. 4,356,196; hereby incorporated by reference). Liquid ammonia impregnates the plant material, which is explosively released upon being exposed upon rapid pressure release. The resulting processed material is used for ethanol production or as a feedstock for food or dairy animals. AFEX processes are also described in European Patent No. 0 077 287; Dale, B. E., et al., Biotech. and Bioengineering Symp. No. 12, 31-43 (1982); Dale, B. E., et al., Developments in Industrial Microbiology, 26 (1985); Holtzapple, M. T., et al. Applied Biochem. and Biotech. 1991, 28/29, 59-74; Blasig, J. D., et al., Resources, Conservation and Recycling 1992, 7, 95-114; Reshamwala, S., et al. Applied Biochem. and Biotech. 1995, 51/52, 43-55; Dale, B. E., et al. Bioresource Tech. 1996, 56, 111-116; and Moniruzzaman, M., et al., Applied Biochem. and Biotech. 1997, 67, 113-126; all of which are incorporated by reference. Pretreatment of biomass using ammonia impregnation typically involves a number of steps. Vaporized ammonia may be recycled in a low pressure vessel.

In certain embodiments, sulfur dioxide-catalyzed steam explosion pre-treatment processes can also be employed using a multi-step protocol. The sulfur dioxide may also be recycled. In certain embodiments, the lignocellulosic materials may be soaked in water or other suitable liquid(s) prior to the addition of steam or ammonia or both, or steam or sulfur dioxide or both. In certain embodiments, the excess water may be drained off the lignocellulosic materials. In certain embodiments, the soaking may be done prior to conveying into a reactor, or subsequent to entry (i.e., inside a pretreatment reactor).

In certain embodiments, ultrasound treatments may be applied to processes of the present invention. See U.S. Pat. No. 6,333,181, which is hereby incorporated by reference.

Steam-explosion has been identified as a low cost and high yield technology, along with low-pressure steam autohydrolysis. Steam explosion heats wetted lignocellulose to high temperatures (e.g., about 160° C. to about 230° C.) and releases the pressure immediately. Rapid decompression flashes the water trapped in the fibers, which leads to a physical size reduction. The elevated temperatures remove acetic acid from hemicellulose which allows some autohydrolysis of the biomass. In certain embodiments, additional chemical agents, such as sulfuric acid or ammonia (e.g., gaseous, anhydrous liquid, or ammonium hydroxide), may be added to aid in the hydrolysis. In certain embodiments, the pretreated cellulose can then be sterilized to prevent growth of other microorganisms during the fermentation reaction.

In certain further embodiments the pre-treatment is a combination of steam treatment and heat treatment. In certain embodiments of the steam treatment and hydrolysis, lignocellulosic biomass is subjected to steam pressure of between 100 psig and 700 psig. A vacuum may be pulled within the reactor to remove air, for example, at a pressure of about 50 to about 300 mbar. Steam may be added to the reactor containing the lignocellulosic material at a saturated steam pressure of between about 100 psig and about 700 psig. More preferably, a saturated steam pressure from about 140 psig to about 300 psig can be used. The temperature of the heat treatment can be about 165° C. to about 220° C. More specifically, the temperature can be about 175° C. to about 210° C., or about 180° C. to about 200° C.

The resultant carbohydrate mixture produced from pre-treatment methods can be further converted to monosaccharides using acid hydrolysis, enzyme hydrolysis or microbes. Microbial hydrolysis produces cellular biomass (single-cell protein) and metabolic waste products, such as organic acids, whilst acid hydrolysis, although simple, produces many additional degradation products, however enzymatic hydrolysis by such enzymes as cellulases, endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, isoamylases provide the cleanest and most preferred approach. Such saccharification enzymes which perform hydrolysis may be produced synthetically, semi-synthetically, or biologically including using recombinant microorganisms.

In certain embodiments of the present invention fermentation organisms can be selected from bacteria, fungi, yeast or a combination thereof. In certain embodiments, useful organisms for biological conversion can include Escherichia, Zymomonas, Saccharomyces, Candida, Pichia, Streptomyces, Bacillus, Lactobacillus, and Clostridium. For example, a recombinant organism selected from the group consisting of Escherichia coli, Zymomonas mobilis, Bacillus stearothermophilus, Saccharomyces cerevisiae, Clostridia thermocellum, Thermoanaerobacterium saccharolyticum, Pichia stipitis, can be added to the reaction solution. In certain embodiments the recombinant organism may perform hydrolysis and fermentation concurrently.

In certain embodiments of the present invention, lignocellulosic pre-treatments occur at higher temperature, longer residence time, and lower pH to initiate a greater extent of hydrolysis, which typically reduces the additional enzyme loading required to liberate soluble monomers that can be metabolized by the organisms responsible for ethanol production. However, mild pre-treatments typically output more carbohydrate oligomers, therefore requiring higher enzyme loading to liberate soluble monomers suitable for conversion.

“Fermentation” or “fermentation process” refers to any process comprising a fermentation step. A fermentation process of the invention includes, without limitation, fermentation processes used to produce alcohols, organic acids, ketones, amino acids, gases, antibiotics, enzymes, vitamins and hormones. Fermentation processes also include fermentation processes used in the consumable alcohol industry, dairy industry, leather industry and tobacco industry. The product of the fermentation process is referred to herein as beer.

In certain embodiments the carbohydrate mixture is further converted to beer via a fermentation step, which contains ethanol and non-fermented solids, which are both recovered. Therefore in certain embodiments of the process of the present invention converting is chemically converting or biologically converting said reactive carbohydrate mixture to form a beer. In certain embodiments chemical conversion comprises acid hydrolysis, alkali hydrolysis, organic solvent treatment or combinations thereof. In certain embodiments biologically converting the reactive carbohydrate mixture to form a beer comprises the addition of bacteria, fungi, yeast or a combination thereof.

In certain embodiments the bacteria, or yeast can be selected from Saccharomyces cerevisiae, Saccharomyces carlsbergensis, Brettanomyces sp., Saccharomyces pastorianus., Pichia spp., Thermoanaerobacter sp., Zymomonas sp., and combinations thereof.

In certain embodiments of the present invention, activated carbon in the free form can be added directly to the lignocellulose feedstock, in some father embodiments of the present invention activated carbon in the free form can be added directly to the reactive carbohydrate mixture. During the degradation of the lignocellulosic structure, not only fermentable sugars are released, but a broad range of compounds, some of which can inhibit the effectiveness of the microorganism used for fermenting. The amount and nature of inhibiting compounds depends on the raw material, the pre-treatment and hydrolysis procedures, and the extent of recirculation in the process.

Fermentation inhibitors in lignocellulosic hydrolysates can be divided into several groups depending on their origin. Substances released during pretreatment and hydrolysis include acetic acid, which is released when the hemicellulose structure is degraded and extractives.

Furthermore, inhibitors, such as furfural and 5-hydroxymethyl furfural, are often produced as by-products in pretreatment and hydrolysis due to the degradation of sugars. Moreover, lignin degradation products are often produced during pretreatment and hydrolysis. This group of inhibitors includes a wide range of aromatic and polyaromatic compounds with a variety of substituents. As such, the addition of activated charcoal in the free form can be used to remove such compounds which inhibit microorganisms and fermentation.

High solids fermentation is particularly prone to long lag phases prior to the onset of fermentation, therefore the addition of activated carbon (charcoal) can reduce or eliminate said lag phase by adsorbing inhibitors, examples of such inhibitors include but are not limited to aldehydes and phenolic compounds.

Activated carbon can be granulated (GAC) or powdered (PAC), traditionally, activated carbons are made in particular form as powders or fine granules less than about 1.0 mm in size with an average diameter between about 0.15 and about 0.25 mm. Thus they present a large internal surface with a small diffusion distance, whilst PAC is made up of crushed or ground carbon particles. Activated carbon of the present invention can display a particle size (mesh) of about 4 to about 325, a surface area of about 600 m²/g to about 1500 m² g and a pore volume of about 0.95 m²/g to about 2 m²/g. In a further embodiment the activated carbon of the present invention can have a plurality of pore sizes, pore volumes and pore surface areas sufficient to selectively adsorb inhibitors having molecular diameters from about 4 Angstroms to about 4000 Angstroms.

In certain embodiments of the process of present invention further comprises: separating activated carbon and solids remaining after pre-treating or solids remaining after said conversion. In some further embodiments separating of said activated carbon and solids is selected from the group consisting of beer column tray separation, paddle dryer apparatus separation, twin screw conveyer separation, rotary valve separation, butterfly valve separation, distillation, centrifuging and combinations thereof.

Certain embodiments of the process of the present invention further comprises de-watering, drying directly or indirectly, and pressing said activated carbon and solids to form a dried solid cake.

There is a need for de-watering of cellulosic fermentation residue because most boiler configurations cannot accept a fuel stream with high moisture content, and moisture naturally decreases the efficiency of the boiler as a portion of the energy released by lignin combustion is absorbed to vaporize the water. Separating the solids from the beer prior to ethanol recovery involves dewatering in a screw press, which is followed by drying. However, the presence of the alcohol during solids separation complicates the drying process, requiring costly and complex closed-loop dryers and with a vapor recovery system. U.S. Pat. No. 4,952,504 (incorporated by reference) discloses that equipment, such as a screen centrifuge or screw press, can be used to de-water solids prior to fermentation.

De-watering prior to fermentation, however, results in loss of fermentable sugars and carries a high capital cost. U.S. Pat. No. 4,552,775 (incorporated by reference) discloses a method for dewatering a stillage comprising 20-30% solids derived from a unique fermentation process. This high solids stillage is combined with sufficient recycled dry product to obtain a 50-70% solids content, which is then pelletized and air-dried.

The recovery and retention of the solids stream allows for the production of the dense, activated carbon rich by-product that can be compressed into energy-rich pellets or dried carbon cakes. Such solids, pellets and cakes are suitable for combustion in various boiler types such as: a fluidized bed boiler; stoker; or suspension fired boilers depending on the degree of de-watering the solids have been subjected to.

In certain embodiments, the heat source used during ethanol stripping and de-watering is direct. In another embodiment, the heat source is indirect. Heat sources include but are not limited to direct steam, direct superheated steam, and indirect steam.

In certain embodiments involving indirect heat sources, the beer can be fed to a paddle dryer apparatus. The agitation provided by the paddle assembly dis-aggregates the beer and conveys it through the vessel as a thin layer of solids in a helical flowpath along the jacketed wall. This enhances mass transfer of volatile materials, ideal for removing tightly entrapped volatiles in materials with fine particle size or poor flowability. The paddles minimize the build-up of solids in order to maintain a high heat transfer rate. These factors combined result in high heat transfer coefficients. This configuration is advantageous because it avoids the risks of plugging or fouling present in the traditional beer column tray and re-boiler design.

In certain embodiments involving direct heat sources, beer is fed to a dryer to which steam or super-heated steam is added. This dryer can be a vessel with positive motion provided by an augur or paddle, or it may be a more complex closed-loop drying system. In the former case, the configuration is as outlined for indirect heating. The beer is fed to a paddle dryer apparatus in which mixing and dis-aggregation is enabled by a paddle assembly; ethanol-water vapor stream is bled from the apparatus. In the latter case, superheated steam dryers are used to deliver heat to the solids and the moisture content to be evaporated. Heat from the superheated steam is transferred to the cooler product as it passes through a duct sized for a particular exposure time. This heat vaporizes a portion of the moisture in the solids, and a bleed stream is constantly drawn from the loop to maintain pressure. The water and ethanol vapor in this bleed stream are discharged from the vessel and passed to a distillation column where ethanol and water are separated without the presence of insoluble solids. This configuration is advantageous because it efficiently dries the solids and allows for vapor recovery of the ethanol.

In some further embodiments involving indirect heat sources, feed material is either pumped or conveyed into a paddle dryer apparatus. The agitation provided by the paddle assembly delumps and conveys the product material through the vessel as a thin-layer of solids in a helical flow-path along the jacketed wall, resulting in very high heat transfer coefficients. The paddles minimize the build-up of solids in order to maintain a high heat transfer rate and to mix and frequently to transport the solids. Drying is established from a heated surface in contact with the product. As the solids are spiraled along the inside vessel wall, heat is transferred by conduction. The water and ethanol vapor stream is discharged from the vessel and passed to a distillation column where ethanol and water are separated without the presence of insoluble solids.

The insoluble solids are then discharged or pushed through the vessel and dried as the water and ethanol are stripped. In another aspect of the invention, cellulosic beer may also contain soluble pentose and hexose sugars that fermentation organisms are unable to metabolize. In one embodiment, the viscous soluble and insoluble solids are discharged through a twin screw conveyor, rotary valve or a double butterfly valve to a cooling belt where they are solidified and mixed with sawdust to produce a stream that can be fed to a process for dense, energy-rich pellet production.

Therefore, in some further embodiments of the process of the present invention, separating of said activated carbon and solids is selected from the group consisting of beer column tray separation, paddle dryer apparatus separation, twin screw conveyer separation, rotary valve separation, butterfly valve separation, distillation, centrifuging and combinations thereof.

In other embodiments the process of the present invention further comprises combusting said dried solid cake to provide heat during production of said ethanol from said lignocellulosic biomass.

In certain embodiments of the process, said converting comprises hydrolyzing cellulose and hemi-cellulose to form monomeric sugars, oligosaccharides or combinations thereof and fermenting said monomeric sugars, oligosaccharides or combinations thereof to produce ethanol.

In some further embodiments of the present invention, hydrolyzing comprises enzymatically hydrolyzing cellulose and hemi-cellulose to form monomeric sugars.

In some further embodiments, said hydrolyzing comprises chemically hydrolyzing cellulose and hemi-cellulose to form monomeric sugars.

In other embodiments, hydrolysis and fermentation take place in separate vessels. Therefore in certain embodiments of the process of the present invention, said fermenting comprises enzymatically fermenting said monomeric sugars to produce ethanol.

In certain embodiments, said hydrolyzing and fermenting occur concurrently in the same reactor. In such cases, one or more aforementioned hydrolysis (saccharification) enzymes may be included in the solution containing one or more of the aformmentioned fermentation organisms.

In some further embodiments of the present invention hydrolyzing and fermenting are concurrent and occur in the presence of activated carbon in free form and in some further embodiments, said activated carbon is granulated or powdered.

In certain embodiments of the present invention said dried solid cake comprises about 1% to about 30% activated carbon and in certain embodiments the process of present invention further comprises burning said dried solid cake as a fuel, wherein said fuel contains about 1 BTU per kilogram to about 16,500 BTU per kilogram. In certain embodiments of the present invention, said dried solid cake comprises activated carbon, lignin, cellulosic sugars, the fermenting organism, ethanol, water and combinations thereof.

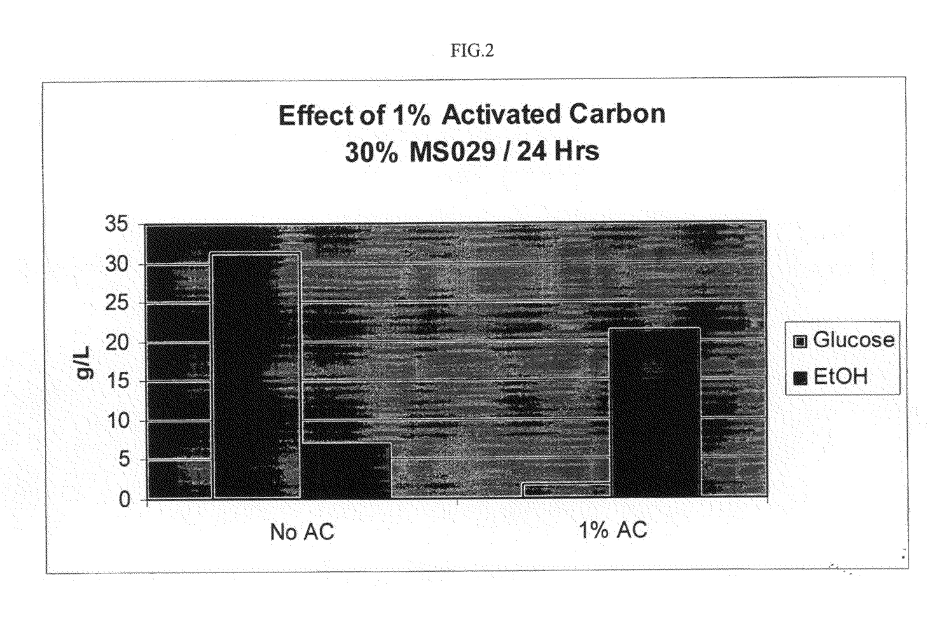

In further embodiments of the present invention, the addition of about 1% to about 6% activated carbon in free form increases the amount of ethanol produced by said process about 50% to about 200% in about 24 hours. The non-limiting example provided below illustrate an example of the process used to produce ethanol from lignocellulose. It can be seen from the graph provided in FIG. 2 that the addition 1% activated carbon in the free form, clearly increases the amount of ethanol produced via the concurrent hydrolysis and fermentation of pre-treated lignocellulose substrate MS029. (also referred to as Simultaneous Saccharification Fermentation (SSF)) of Example 1.

### EXAMPLES

### Example 1

TABLE 1
A standard high solids (30%) SSF process

| | |
|---|---|
| 1 | Autoclave empty fermentor for 30 min, add: |
| | 402.57 g MS029 substrate (pre-treated at 160 psi for 10 minutes) |
| | 7 ml 5M KOH |
| | 100 ml 10X YP (yeast extract and peptone) |
| | 5 mM MgSO₄ |
| | 25 mg Spezyme CP/g ODS (35.46 ml) (cellulase-breaks |
| | down oligosaccharides) |
| | 15 mg Novozyme 188/g ODS (12.8 ml) (cleaves |
| | β-glucosidase to glucose) |
| | 107.73 ml DIH₂O |
| 2 | Incubate @ 50° C., 500 rpm for 2 hrs, add: |
| | 201.28 g MS029 |
| | 3.2 ml Novozyme 188 |
| | *10 g Activated Carbon (Sigma # 242268) |
| 3 | Incubate at 50° C., 500 rpm for 1 hr |
| 4 | Reduce temp. To 30° C., agitation to 250 rpm |
| | Add 3 mg penicillin G (Sigma # P7794) |
| 5 | Inoculate (10% V/V) and ferment |

These examples illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A process of producing ethanol from lignocellulosic biomass, which comprises:
pre-treating a lignocellulosic feedstock to produce a reactive carbohydrate mixture;
adding activated carbon in free form;
converting said reactive carbohydrate mixture to form a beer;
separating solids from said carbohydrate mixture or said beer or both,
wherein said activated carbon is separated along with the solids in said mixture, said beer or both; and
drying said solids.

2. The process of claim 1, wherein said converting is chemically converting or biologically converting said reactive carbohydrate mixture to form a beer.

3. The process of claim 1, which further comprises: separating activated carbon and solids remaining after pre-treating or solids remaining after said biological conversion.

4. The process of claim 3, wherein the process of separating said activated carbon and solids is selected from the group consisting of beer column tray separation, paddle dryer apparatus separation, twin screw conveyer separation, rotary valve separation, butterfly valve separation, distillation, centrifuging and combinations thereof.

5. The process of claim 4, which further comprises de-watering, drying directly or indirectly, and pressing said activated carbon and solids to form a dried solid cake.

6. The process of claim 5, which further comprises combusting said dried solid cake to provide heat during production of said ethanol from said lignocellulosic biomass.

7. The process of claim 1, wherein said lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof.

8. The process of claim 1, wherein said pre-treating is selected from the group consisting of catalytic treatment, acid treatment, alkaline treatment, organic solvent treatment, steam treatment, heat treatment, low-pH treatment, pressure treatment, milling treatment, steam explosion treatment, pulping treatment or white rot fungi treatment and combinations thereof.

9. The process of claim 8, wherein the pre-treatment is a combination of steam treatment and heat treatment.

10. The process of claim 2, wherein said converting comprises hydrolyzing cellulose and hemi-cellulose; to form monomeric sugars; and fermenting said monomeric sugars to produce ethanol.

11. The process of claim 10, wherein said hydrolyzing comprises enzymatically hydrolyzing cellulose and hemi-cellulose to form monomeric sugars.

12. The process of claim 10, wherein said hydrolyzing comprises chemically hydrolyzing cellulose and hemi-cellulose to form monomeric sugars.

13. The process of claim 10, wherein said fermenting comprises enzymatically fermenting said monomeric sugars to produce ethanol.

14. The process of claim 10, wherein said hydrolyzing and fermenting occur concurrently in the same reactor.

15. The process of claim 10, wherein hydrolyzing and fermenting are concurrent and occur in the presence of activated carbon in free form.

16. The process of claim 1, wherein said activated carbon is granulated or powdered.

17. The process of claim 5, wherein said dried solid cake comprises about 1% to about 30% activated carbon.

18. The process of claim 5, which further comprises burning said dried solid cake as a fuel, wherein said fuel contains about 1 BTU per kilogram to about 16,500 BTU per kilogram.

19. The process of claim 1, whereby the addition of about 1% to about 6% activated carbon in free form increases the amount of ethanol produced by said process about 50% to about 200% in about 24 hours.

20. The process of claim 5, wherein said dried solid cake comprises activated carbon, lignin, cellulosic sugars, ethanol, water and combinations thereof.
